(19) United States
(12) Patent Application Publication
(10) Pub. No.: US 2021/0237058 A1
(43) Pub. Date: Aug. 5, 2021

(54) MICROFLUIDIC DEVICES TO DETECT FLUID PRIMING

(71) Applicant: HEWLETT-PACKARD DEVELOPMENT COMPANY, L.P., Spring, TX (US)

(72) Inventors: James Michael Gardner, Corvallis, OR (US); Daryl E. Anderson, Corvallis, OR (US); Eric Martin, Corvallis, OR (US); Alexander Govyadinov, Corvallis, OR (US)

(73) Assignee: HEWLETT-PACKARD DEVELOPMENT COMPANY, L.P., Spring, TX (US)

(21) Appl. No.: 17/049,287

(22) PCT Filed: Aug. 9, 2018
(86) PCT No.: PCT/US2018/046090

§ 371 Date: Oct. 20, 2020

(51) Int. Cl. B01L 3/00; G01N 27/02

(52) U.S. Cl. CPC B01L 3/502715; G01N 27/02; B01L 2200/027; B01L 2300/0627; B01L 2200/06; B01L 2400/0475

(57) ABSTRACT

A microfluidic device may include an impedance sensor located within a fluidic priming orifice within a fluidic channel of the microfluidic device, and control logic. The control logic is to force a current into the impedance sensor to sense the presence of a fluid within the fluidic channel at the locations of the impedance sensor, and determine if the fluid is primed into the fluidic channel based on the impedance values sensed by the impedance sensor.

### BACKGROUND

Microfluidics, as it relates to the sciences, may be defined as the manipulation and study of minute amounts of fluids, and microfluidics devices may be used in a wide range of applications within numerous disciplines such as engineering, physics, chemistry, biochemistry, nanotechnology, and biotechnology along with other practical applications. Microfluidics may involve the manipulation and control of small volumes of fluid within various systems and devices such as lab-on-chip devices, printheads, and other types of microfluidic chip devices.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a microfluidic device, according to an example of the principles described herein.

FIGS. 2A through 2C are block diagrams of a microfluidic device at various levels of passive priming, according to an example of the principles described herein.

FIG. 3A is a block diagram of a microfluidic device including sensors to detect single, dual, or differential passive priming sense, according to an example of the principles described herein.

FIG. 3B is a block diagram of a microfluidic device including sensors to detect a high change of on/off resistance, according to an example of the principles described herein.

FIG. 3C is a block diagram of a microfluidic device including sensors to detect a priming flow rate, according to an example of the principles described herein.

FIGS. 4A through 4C are block diagrams of a microfluidic device at various levels of active priming, according to an example of the principles described herein.

FIG. 5A is a block diagram of a microfluidic device including sensors to detect single, dual, or differential active priming sense, according to an example of the principles described herein.

FIG. 5B is a block diagram of a microfluidic device including sensors to detect a boundary of fluid within a passageway of the microfluidic device, according to an example of the principles described herein.

FIG. 6A is a block diagram of a microfluidic device, according to an example of the principles described herein.

FIG. 6B is a block diagram of a microfluidic device including an extended sensor, according to an example of the principles described herein.

FIG. 7 is a flowchart showing a method of detecting priming of a microfluidic device, according to an example of the principles described herein.

FIG. 8 is a flowchart showing a method of detecting priming of a microfluidic device, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Microfluidic devices may enable manipulation and control of small volumes of fluid through microfluidic fluidic channels or networks of the microfluidic devices. For example, microfluidic devices may enable manipulation and/or control of volumes of fluid on the order of microliters (i.e., symbolized μL and representing units of 10⁻⁶ liter), nanoliters (i.e., symbolized nL and representing units of 10⁻⁹ liter), picoliters (i.e., symbolized pL and representing units of 10⁻¹² liter) or femtoliters (i.e., symbolized fL and representing units of 10⁻¹⁵ liter). Thus, microfluidic devises process low volumes of fluids to achieve multiplexing, automation, and high-throughput screening.

Microfluidic devices employ sensors such as, for example, biosensors, bioelectrical sensors, cell-based sensors, and other sensors that provide point of care diagnostics for medical diagnostics, food analysis, environmental monitoring, drug screening and other point of care applications. Cell-based sensor apparatus, for example, detect or measure cellular signals from living cells of a sample fluid to identify, for example, a specific species of bacteria, virus and/or disease. In operation, as the fluid flows adjacent, past or across the sensors such as electrodes, the sensors detect or convert signals detected in the fluid to electrical signals that are analyzed to determine or identify a characteristic of the fluid detected by the sensor. For example, the sensors may employ an electrode positioned in a fluidic channel or a sensor chamber, and an interaction between the fluid and a surface of the electrode may be monitored by applying a signal such as a voltage or current for a period of time, and observing a resulting signal such as a current or voltage on the electrode. The resulting signal may be sensed at the same or a different electrode as the one to which the activation signal was applied. In some examples, the fluid has a different impedance versus empty space with only air present, and the difference in impedance of the fluid versus the air alters the voltage on the electrode.

In microfluidic systems, such as lab-on-chip (LOC) designs for molecular diagnostics, ensuring complete fluidic priming of the device allows the microfluidic devices to perform their functions as diagnostic devices. Either passive fluid priming methods such as capillary action, or active fluid-priming methods such as pumping are used depending on the complexity of the system and its deployment strategy and use case. Capillary action is the ability of a liquid to flow in narrow spaces without the assistance of an external power source, or even in opposition to, external forces such as gravity. Pumping may use a pump or other pressure differential device to create pressure within a microfluidic channel to move the fluid through the microfluidic channel. In both passive and active fluid priming methods, with the small-scale system of a microfluidic device, it may be desirable to have in-situ detection of fluid presence at locations throughout the microfluidic system.

Using electrical impedance sensors distributed within in a microfluidic device, detection of fluid fill during priming of at least one channel, reservoir, chamber, or other passageway of the microfluidic device, the presence of unwanted air in the system that may adversely affect measurement results, and an end of fluid reservoir volume detection may be provided throughout the functioning of the microfluidic device. Further, a method may be executed whereby the fluidic priming may be monitored from the inlet to outlet and throughout the microfluidic device before, during, and after, diagnostic testing of the fluid begins. In cases of priming failure, the microfluidic device may provide feedback to the system to correct the fluidic priming issue. Feedback of fluid presence in the various passageways of the microfluidic device provides in-situ smart sensing capabilities to improve success of LOC applications.

Examples described herein provide a microfluidic device. The microfluidic device may include an impedance sensor located within a fluidic priming orifice within a fluidic channel of the microfluidic device, and control logic. The control logic forces a current or voltage into the impedance sensor to sense the presence of a fluid within the fluidic channel at the locations of the impedance sensor, and determines if the fluid is primed into the fluidic channel based on the impedance values sensed by the impedance sensor. In one example, the current or voltage may be forced into a first impedance sensor and detected by a second impedance sensor located near the first impedance sensor.

The control logic monitors for at least a second time the impedance sensed at the impedance sensor. Further, the control logic determines if the impedance sensor detects the absence of fluid in the fluidic channel once the fluid has been detected as primed into the fluidic channel. The absence of the fluid indicates a failed priming of fluid into the fluidic channel. The control logic sends an activation signal to correct the failed priming of fluid into the fluidic channel. The activation signal to correct the failed priming of fluid into the fluidic channel may include a signal to cause a pumping device to move fluid into the fluidic channel or issue a notification for further user intervention.

The impedance sensor may include a single impedance sensor. The single impedance sensor may include a conductive plate with a length of at least a portion of the fluidic channel. The single impedance sensor, when actuated, provides an analog signal that correlates with an amount of fluid within the fluidic channel. The control logic determines when the priming of fluid into the fluidic channel is complete, and, in response to a determination that the priming of fluid into the fluidic channel is complete, instructs the processing of the fluid within the microfluidic device.

Examples described herein provide a system for detecting priming of a microfluidic device. The system may include a fluidic channel priming detection array including at least one impedance sensor located within a fluidic channel of the microfluidic device, and control logic. The control logic forces a current into the impedance sensor to sense the presence of a fluid within the fluidic channel, determines if the fluidic channel is primed based on the impedance values sensed by the impedance sensor, and sends an activation signal to correct a failed priming of fluid into the fluidic channel in response to a determination that the fluidic channel is not primed.

Further, the control logic monitors the impedance sensed about the impedance sensor at least a second time, and determines if the impedance sensor detects the absence of fluid in the fluidic channel once the fluid has been detected as primed into the fluidic channel. The absence of the fluid indicates a failed priming of fluid into the fluidic channel. The activation signal to correct the failed priming of fluid into the fluidic channel may include a signal to cause a pumping device to move fluid into the fluidic channel via the orifice or issue a notification for further user intervention.

The at least one impedance sensor of the fluidic channel priming detection array may include a first impedance sensor located at a first position at a fluidic priming orifice within a fluidic channel of the microfluidic device, and a second impedance sensor located at a second position within the fluidic priming orifice within the fluidic channel downstream relative to the first impedance sensor. The first impedance sensor and the second impedance sensor detect the presence of the fluid at relative locations of the first impedance sensor and the second impedance sensor. The at least one impedance sensor of the fluidic channel priming detection array may include a conductive plate with a length of at least a portion of the fluidic channel. The single impedance sensor, when actuated, provides an analog signal that correlates with an amount of fluid within the fluidic channel. The control logic may determine when the priming of fluid into the fluidic channel is complete, and, in response to a determination that the priming of fluid into the fluidic channel is complete, instruct the processing of the fluid within the microfluidic device.

Examples described herein provide a method of detecting priming of a microfluidic device. The method may include forcing a current into an impedance sensor of a fluidic channel priming detection array to sense the presence of a fluid within the fluidic channel. The impedance sensor is located within a fluidic channel of the microfluidic device. The method also includes determining if the fluidic channel is primed based on the impedance values sensed by the impedance sensor.

The method may also include continuously monitoring the impedance sensed at the impedance sensor, and determines if the impedance sensor detects the absence of fluid in the fluidic channel once the fluid has been detected as primed into the fluidic channel. The absence of the fluid indicates a failed priming of fluid into the fluidic channel. The method may also include sending an activation signal to correct the failed priming of fluid into the fluidic channel. The activation signal may include a signal to cause a pumping device to move fluid into the fluidic channel via the orifice. The method may also include determining when the priming of fluid into the fluidic channel is complete, and, in response to a determination that the priming of fluid into the fluidic channel is complete, instructing the processing of the fluid within the microfluidic device.

As used in the present specification and in the appended claims, the terms “passive” in the context of priming passageways within a microfluidic device is meant to be understood broadly as any process used to prime the passageways that does not use the application of a pressure differential applied to an end of the passageway to move the fluid through the passageway. In one example, passive priming of the passageway may be achieved through capillary forces where a fluid flows in the passageways without the assistance of, or even in opposition to, external forces such as gravity. Capillary action occurs because of intermolecular forces between the fluid and surrounding solid surfaces such as the interior surfaces of the passageways defined within the microfluidic device. If the diameter or cross-section of the passageways are sufficiently small, then the combination of surface tension caused by cohesion within the fluid and adhesive forces between the fluid and the walls of the passageways may produce high enough pressure to propel the fluid.

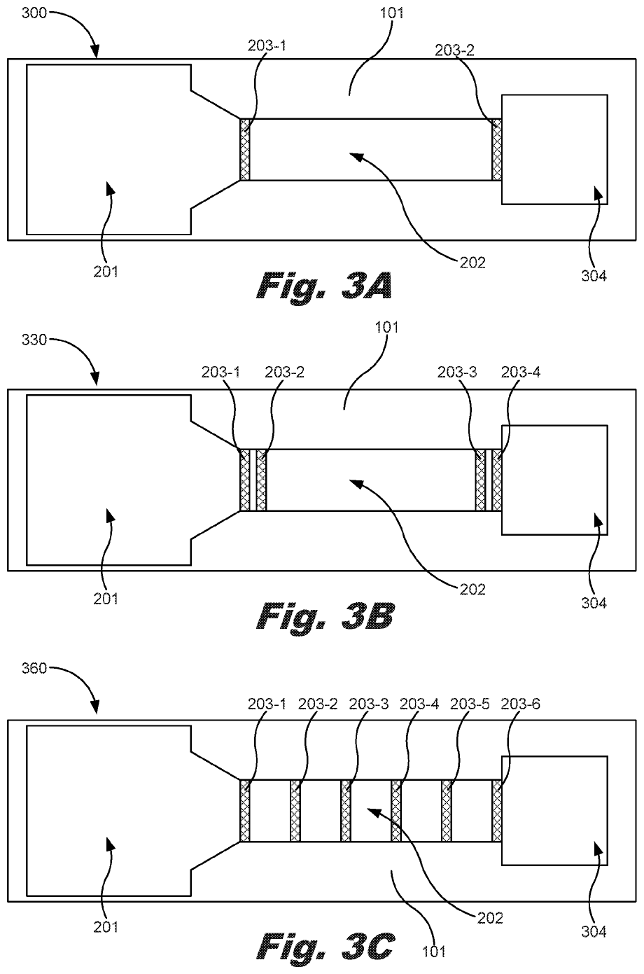

As used in the present specification and in the appended claims, the terms “active” in the context of priming passageways within a microfluidic device is meant to be understood broadly as any process used to prime the passageways that uses the application of a pressure differential applied to the fluid to move the fluid through the passageway. In one example, active priming of the passageway may be achieved through activation of a pump device. The pump devices may include inertial pumps that include thermal resistive elements or piezoelectric elements, or external pump devices that are fluidically coupled to an end of the passageway such as the reservoir (201), the nozzle chamber (204), or terminating chamber (FIGS. 3A-3C, 304) to create a positive or negative pressure within the passageways to move the fluid through the passageways.

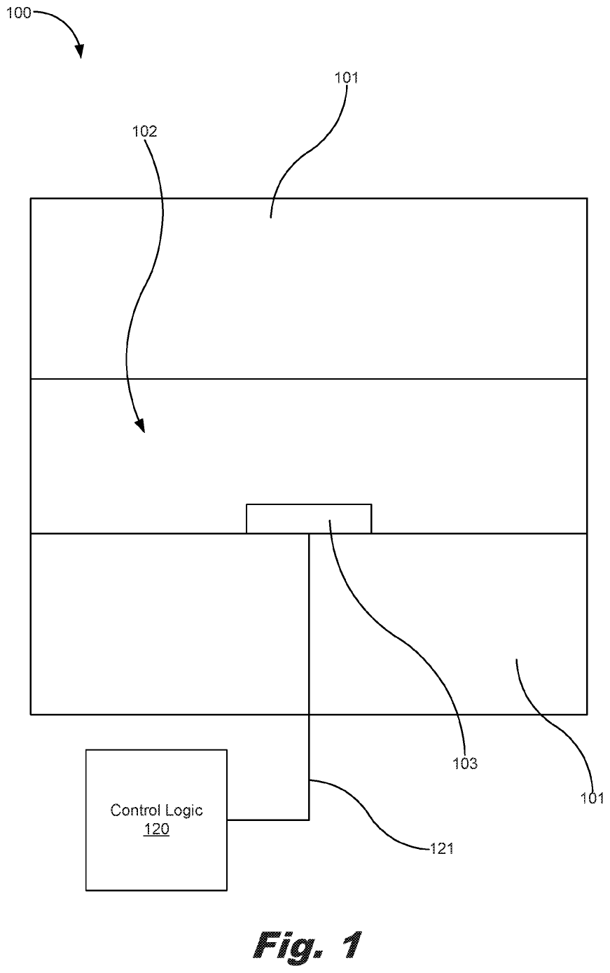

Turning now to the figures, FIG. 1 is a block diagram of a microfluidic device (100), according to an example of the principles described herein. The elements of the microfluidic devices and their functions and purposes described herein may be used in any type of microfluidic device including, for example, assay systems, point of care systems, and any systems that involve the use, manipulation, control of small volumes of fluid, and combinations thereof. For example, the microfluidic device (100) may incorporate components and functionality of a room-sized laboratory or system to a small chip such as a microfluidic biochip or “lab-on-chip” (LOC) that manipulates and processes solution-based samples and systems by carrying out procedures that may include, for example, mixing, heating, titration, separation, other chemical and physical processes, and combinations thereof. For example, the microfluidic device (100) may be used to integrate assay operations for analyzing enzymes and DNA, detecting biochemical toxins and pathogens, diagnosing diseases, viruses, and bacteria, other chemical and biochemical processes, and combinations thereof.

The microfluidic device (100) may include a die (101) with at least one microfluidic passageway (102) defined therein. The die (102) may be made of, for example, silicon (Si). In an example, the die (101) may include a plurality of microfluidic passageways (102) defined therein in any configuration and architecture to provide for the movement, mixing, and reacting of fluids within the microfluidic device (100). The microfluidic passageways (102) may include fluidic inlets, reservoirs, chambers, reactors, reaction cites, junctions, channels, capillary breaks, outlets, nozzles, venting ports, drains, and other architectures for use in accomplishing the desired chemical and physical processes of the microfluidic device (100).

At least one impedance sensor (103) is located within a microfluidic passageway (102) of the microfluidic device (100). In one example, the impedance sensor (103) may be located at a fluidic priming orifice within a fluidic channel of the microfluidic device (100). The at least one impedance sensor (103) may be any device that can sense an impedance value of a fluid within the microfluidic passageways (102). In one example, the impedance sensor (103) may be an electrode electrically coupled to a voltage or current source. The electrode may be a thin-film electrode formed on an interior surface of the microfluidic passageways (102) defined within the die (101) of the microfluidic device (100). In an example, a current may be applied to the impedance sensor (103) when a priming of the microfluidic passageway (102) is to be detected, and a voltage may be measured. In another example, a voltage may be applied to the impedance sensor (103) when a priming of the microfluidic passageway (102) is to be detected, and a current may be measured. In an example, the impedance sensors (103) described herein may include a tantalum (Ta), Aluminum (Al), or Gold (AU) sensor plate electrically coupled to control logic (120) by an electrical line (121).

In an example, the impedance sensors (103) may positioned throughout the microfluidic device (100) where they may contact a fluid that is subjected to testing within the microfluidic device (100). Placing impedance sensors (103) near the fluidic priming inlets and outlets allows for the microfluidic device (100) to confirm a priming of filling of the fluid in the passageways of the microfluidic device (100), or a reservoir depletion during micro-titration or other physical and chemical processes preformed on the fluid. Further, placing impedance sensors (103) throughout the microfluidic device (100) provides for in-situ fluid priming monitoring and trapped air detection that may be remedied through intervention by the microfluidic device (100).

The control logic (120) may be used to send electrical signals to the impedance sensors (103), receive detected impedance values at the sensors (103), activate a number of internal or external pumps to control the location of the fluid within the passageways of the microfluidic device (100) based on feedback from the sensors (103), activate a number of measurement devices to measure a characteristic or property of the fluid, perform other processes, and combinations thereof. Throughout the present description, the control logic (120) may be coupled to any device within the microfluidic device (100) including impedance sensors and pumps to control the activation of these devices and the receipt of date from the devices. In one example, to measure the impedance at the impedance sensors (103), a small current may be forced into the impedance sensors (103), and a resulting voltage may be measured after a predetermined amount of time. In this example, if the impedance sensors (103) is not in contact with the fluid (i.e., air is surrounding the impedance sensors (103)), the impedance measured may be relatively high compared to if the impedance sensors (103) are in contact with fluid where the impedance measured will be relatively much lower.

In the example where a fixed current is applied to the at least one impedance sensor and dissipated through the fluid surrounding the at least one impedance sensor (103), a resulting voltage may be measured on the conductive plate of the at least one impedance sensor. The sensed voltage may be used to determine an impedance of the fluid, whether it be air or another fluid such as an analyte, surrounding the at least one impedance sensor (103) at that area within the microfluidic passageway (102) at which the at least one impedance sensor (103) is located. Electrical impedance is a measure of the opposition that the circuit formed from the at least one impedance sensor (103) and the fluid presents to a current when a voltage is applied to the impedance sensor (103), and may be represented as follows:

$$Z = \frac{V}{I} \qquad Eq . 1$$

where Z is the complex impedance in ohms (Ω), V is the voltage applied to the impedance sensor (103), and I is the current applied to the impedance sensor (103). In another example, the impedance may be complex in nature, such that there may be a capacitive element to the impedance where the fluid may act partially like a capacitor. For complex impedances, the current applied to the impedance sensor (103) may be applied for a particular period of time, and a resulting voltage may be measure at the end of that time.

The detected impedance (Z) is corresponds to an impedance value of the fluid; whether that fluid is air, vapor, or another fluid being primed into the microfluidic passageway (102). Stated in another way, the impedance (Z) is corresponds to, for example, a dispersion level of the particles, ions, or other chemical and physical properties of the fluids. In one example, if the impedance detected at the impedance sensors (103) is relatively higher, this may indicate that the fluid has a higher impedance in that area at which the impedance is detected. This relatively higher impedance may indicate that the fluid surrounding the impedance sensor (103) is air which, in many cases, may have a higher impedance relative to other fluids such as analytes, and other chemical solutions. Conversely, a relatively lower impedance within that portion of the fluid may indicate that the fluid surrounding the impedance sensor (103) is a fluid other than air which, in many cases, may have a lower impedance relative to air. In this manner, it may be determined that a fluid other than air has not yet made it to that impedance sensor (103), and that at least that portion of the microfluidic passageway (102) has not been primed. In one example, the fluid may be allowed to move further down the microfluidic passageway (102) or may be retained at a position within the microfluidic passageway (102) depending on what the chemical or physical process is being performed within the microfluidic passageway (102). In some examples, it may be desirable to keep the non-air fluid from priming the microfluidic passageway (102) for an amount of time, and then allow the fluid to move further down the microfluidic passageway (102) at a later time.

The control logic (120) of FIG. 1 may be any combination of hardware and computer-readable program code that is executed to force a current into the impedance sensors (103) to sense the presence of a fluid within the microfluidic passageway (102) at the locations of the impedance sensors (103). The control logic (120) also determines if the non-air fluid is primed into the microfluidic passageway (102) based on the impedance values sensed by the impedance sensors (103). The priming of the fluid within at least a portion of the microfluidic passageways (102) of the microfluidic device (100) may be tracked based on sensed impedance at the impedance sensors (103) that is relayed to the control logic (120). Processing devices integrated within or external to the control logic (120) may be used to analyze the sensed impedances and determine a position of the fluid within and a degree of the priming of the fluid within the microfluidic passageway (102) of the microfluidic device (100).

Further, the control logic (120) may monitor for at least a second time or instance, the impedance sensed at the impedance sensor. As the fluid is passively or actively primed into the microfluidic passageway (102), it may be desirable to ascertain the progress of the priming of the fluid within the microfluidic passageway (102) and to determine a flow rate of the fluid using a plurality of impedance sensors (103). Still further, the control logic (120) may determine if the impedance sensors (103) detect the absence of fluid in microfluidic passageway (102) once the fluid has been detected as primed into the microfluidic passageway (102). In one example, the absence of the fluid indicates a failed priming of fluid into the microfluidic passageway (102) such as the introduction of an air bubble into the microfluidic passageway (102) or a depletion of the fluid that is sourced into the microfluidic passageway (102) from, for example, a reservoir. In this example, the control logic (120) may send an activation signal to correct the failed priming of fluid into the microfluidic passageway (102). The activation signal used to correct the failed priming of fluid into the microfluidic passageway (102) may include a signal to cause a pumping device to move fluid into the fluidic channel.

In an example, the microfluidic device (100) detects air bubble formation and an end of the fluid in the microfluidic device (100). Detecting the end of the fluid within the microfluidic passageway (102) may cause the control logic to produce a signal that includes instruction to cause other fluid to be introduced into the microfluidic passage (102). For example, after a first fluid has been processed by the microfluidic device (100), other fluids may be introduced to the microfluidic device (100) for processing once the impedance sensors (103) detect the absence of the first fluid in microfluidic passageway (102). A second fluid such as, for example, washing fluid used to clean the first fluid out of the passageways of the microfluidic device (100) or other fluids used in chemical or biochemical reactions may be introduced into the passageways of the microfluidic device (100) after the exhaustion of the first fluid has been detected by the impedance sensors detecting the end of the first fluid and, instead, air in the microfluidic passageway (102). In this manner, the control logic (120) of the microfluidic device (100) may providing signals to prime the microfluidic passageways (102) with different fluids.

The control logic (120) may also determine when the priming of fluid into the microfluidic passageway (102) is complete. In response to a determination that the priming of fluid into the microfluidic passageway (102) is complete, the control logic (120) may instruct the processing of the fluid within the microfluidic device (100) by activating a number of fluid processing elements within the microfluidic device (100).

In the examples described herein, the impedance sensor (103) may include a single impedance sensor. In this example, the single impedance sensor (103) may include a conductive plate forms a capacitor that produces an analog measurement proportional to how much fluid is in contact with the conductive plate. In another example, the single impedance sensor (103) may include a resistive plate such as a potentiometer with a length of at least a portion of the fluidic channel. The single impedance sensor, when actuated, provides an analog signal that correlates with an amount of fluid within the microfluidic passageways (102).

In the examples described herein, the control logic (120) may activate an internal pump device and/or an external pump device to move the fluid within the at least one microfluidic passageway (102) in an upstream direction, a downstream direction, and combinations thereof. For example, the control logic (120) may receive a signal from the at least one impedance sensor (103) that defines a sensed impedance at the impedance sensor. If an intended outcome is to ensure that the fluid does not pass that impedance sensor (103) and the sensed impedance indicates that the fluid is located at least at the impedance sensor (103), then the control logic (120) may cause a pump device to create a negative pressure on the microfluidic passageways (102) to pull the fluid back upstream past the impedance sensor (103). The control logic (120) may receive a sensed impedance from the impedance sensor (103) that indicates that the fluid has passed the impedance sensor (103) back upstream, and the control logic (120) may instruct the pump device to create a negative pressure sufficient to restrict the fluid form moving past the impedance sensor in the downstream direction again and hold the fluid at its current position.

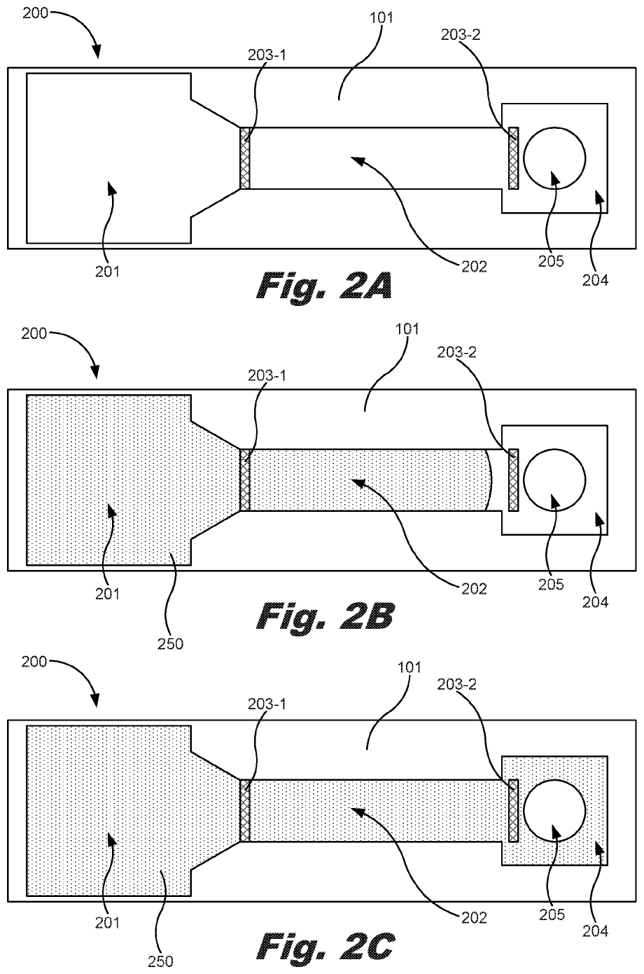

FIGS. 2A through 2C are block diagrams of a microfluidic device (200) at various levels of passive priming, according to an example of the principles described herein. The microfluidic device (200) of FIGS. 2A through 2C include a number of fluidic inlets, reservoirs, chambers, reactors, reaction cites, junctions, channels, capillary breaks, outlets, nozzles, venting ports, drains, and other architectures for use in accomplishing the desired chemical and physical processes of the microfluidic device (200). Specifically, the microfluidic device (200) of FIGS. 2A through 2C may include a reservoir (201) into which a fluid (250) to be analyzed within the microfluidic device (200) is placed to allow the fluid (250) to enter other passageways within the die (101) of the microfluidic device (200). A fluidic channel (202) may be fluidically coupled to the reservoir (201) to allow for the conveyance of the fluid (250) toward a nozzle chamber (204). Although a nozzle chamber (204) with a nozzle (205) defined therein is depicted in FIGS. 2A through 2C, a drain or reaction chamber may be the destination of the fluid (250). The nozzle (205) may be used to eject the fluid (250) from the nozzle chamber (204) into another passageway, another microfluidic device, or out of the microfluidic device (200) entirely. In this manner, the reservoir (201), fluidic channel (202), nozzle chamber (204), and nozzle (205) are fluidically coupled to one another, and the fluid may enter these and other architectures of the microfluidic device (200) through passive capillary forces.

The fluid (250) has not been introduced into the reservoir (201) of the microfluidic device (200) at the state depicted in FIG. 2A. However, at the state of the microfluidic device (200) depicted in FIG. 2B, the fluid (250) has been introduced into the reservoir (201), and the fluid (250), through capillary forces, has traveled into the fluidic channel (202) past a first impedance sensor (203-1). The first impedance senor (203-1) in FIG. 2A will detect that the fluid (250) is not present at and past the first impedance senor (203-1). Likewise, the second impedance senor (203-2) in FIG. 2A will detect that the fluid (250) is not present at and past the second impedance senor (203-2) or within the nozzle chamber (204). The sensors (203) described throughout the figures will be collectively referred to herein as 203.

In FIG. 2B, the first impedance senor (203-1) will detect the fluid (250) has traveled down the fluidic channel (202) at least as far as the location of the first impedance senor (203-1), but the second impedance senor (203-2) will still detect that the fluid (250) has not reached its position or entered the nozzle chamber (204). Thus, the control logic (100), to which all impedance sensors (203) described herein are electrically and communicatively coupled, will determine that the fluid (250) has entered the fluidic channel (202), and not the nozzle chamber (204), and has wicked through capillary forces to a point between the first impedance sensor (203-1) and the second impedance sensor (203-2). In an example, more impedance sensors (203) may be included within the fluidic channel (202) to determine a more precise location of the fluid's (250) capillary progress within the fluidic channel (202).

In FIG. 2C, the fluid has traveled through the reservoir (201), the fluidic channel (202), and into the nozzle chamber (204). In one example, once the second impedance sensor (203-2) detects the fluid (250) within the nozzle chamber (204), the fluid (250) may be ejected out of the microfluidic device (200) through the nozzle through activation of a thermal or piezoelectric ejection device, for example.

Further, as described herein, an external pump may be fluidically coupled to either end of the passageway of the microfluidic device (200) to apply positive or negative pressure in addition to or in place of the capillary forces used to move the fluid (250) within the passageways. In this example, the external pump may cause a negative pressure to be placed on the fluid (250) to cause the fluid to stop its capillary movement through the fluidic channel (202) as depicted in FIG. 2B to retain the fluid at the position between the first impedance sensor (203-1) and the second impedance sensor (203-2) for a period of time. In this example, the fluid (250) may be retained at this position to allow for other physical and chemical reactions to take place within the microfluidic device (200) before the fluid (250) within the microfluidic device (200) is introduced to other portions of the microfluidic device (200).

Further, other devices such as heating devices used to heat the fluid (250), cooling devices used to cool the fluid (250), mixing devices to mix the fluid (250), other devices to change a physical or chemical property of the fluid may also be included within the reservoir (201), fluidic channel (202), nozzle chamber (204), nozzle (205), or any other passageway or architecture of the microfluidic device (200). These additional devices may be included to allow these devices to alter the fluid's (250) physical and/or chemical properties before the fluid is allowed to proceed further within the microfluidic device (200). The external pump may be used to retain the fluid (250) at a position within the microfluidic device (200) to allow these additional devices to perform their respective functions before the fluid (250) is allowed to proceed within the passageways within the microfluidic device (200).

FIGS. 2A through 2C depict the manner in which the fluid (250) may passively move within the passageways of the microfluidic device (200). Once priming of the passageways of the microfluidic device (200) including the reservoir (201), fluidic channel (202), nozzle chamber (204), nozzle (205) as detected by the impedance sensors (203-1, 203-2) is determined to have completed as depicted in FIG. 2C, measurements and/or processing of the fluid (250) may take place.

In an example, the example of FIGS. 2A through 2C may include one impedance sensor (203). In this example, the one impedance sensor (203) may be used to detect one instance in which the fluid (250) passes that one impedance sensor (203). The one impedance sensor (203) may also be able to detect the absence of the fluid (250) after an initial priming of the fluid (250) has occurred or a first detection of the fluid (250) has been made by the one impedance sensor (203). A detection of an absence of the fluid (250) after an initial priming of the fluid (250) has occurred or a first detection of the fluid (250) has been made by the one impedance sensor (203) may be the result of an air bubble introduced into the fluid (250) further upstream, or may result from fluid (250) no longer being introduced into the microfluidic device (200) or a complete consumption of an amount of the fluid (250) by the microfluidic device (200). The one impedance sensor (230) and any other impedance sensor described herein may continually detect whether the fluid (250) is located at its position in the microfluidic device (200) so that any disrupted flow of the fluid (250) may be detected, including disruptions such as, for example, an air bubble or an end of the fluid in the microfluidic device (100).

In another example, at least two impedance sensors (203-1, 203-2) may be used to detect a differential value. For example, in FIG. 2B, the impedance value detected by the first impedance sensor (203-1) may be compared to the value detected by the second impedance sensor (203-2) to determine the location of the fluid (250) within the microfluidic device (200). In FIG. 2B, the first impedance sensor (203-1) may detect a relatively lower impedance value with respect to the impedance value detected by the second impedance sensor (230-2) because the impedance of the fluid (250) surrounding the first impedance sensor (203-1) may have a relatively lower impedance with respect to air which the second impedance sensor (230-2) may be exposed to. This differential value may be used by the control logic (120) to determine that the fluid (250) has passed the first impedance sensor (203-1) but not the second impedance sensor (203-2).

Thus, throughout the examples described herein, in sensing the priming of the fluid (250) within the passageways of the microfluidic device (100, 200, 300, 330, 360, 400, 500, 550, 600, 650) in a singular manner, the control logic (120) may simply send a signal to an impedance sensor (203) to detect whether the fluid (203) is located at that impedance sensor (203) irrespective or independent of the inclusion of another impedance sensor (203) within the microfluidic device (100, 200, 300, 330, 360, 400, 500, 550, 600, 650) or the activation of another impedance sensor (203) to sense an impedance at that other impedance sensor (203).

When used as dual sensors (203) including at least two impedance sensors (203), the control logic (120) may send an activation signal to the at least two impedance sensors (203) and an impedance value may be received by the control logic (120) from each of the two or more impedance sensors (203) to determine the degree of the priming of the fluid (250) within the passageways of the microfluidic device (100, 200, 300, 330, 360, 400, 500, 550, 600, 650).

When a plurality of impedance sensors (203) are used in a differential manner, the control logic (120) may send an activation signal to the plurality of impedance sensors (203) and an impedance value may be received by the control logic (120) from each of the plurality of impedance sensors (203). The impedance values obtained from each of the plurality of impedance sensors (203) may be compared to track a differential signal to determine the degree of the priming of the fluid (250) within the passageways of the microfluidic device (100, 200, 300, 330, 360, 400, 500, 550, 600, 650).

FIG. 3A is a block diagram of a microfluidic device (300) including sensors to detect single, dual, or differential passive priming sense, according to an example of the principles described herein. The microfluidic device (300) includes many elements that are included within the microfluidic device (200) of FIGS. 2A through 2C. The microfluidic device (300) of FIG. 3A, and the microfluidic devices (330, 360) of FIGS. 3B and 3C include a terminating chamber (304) that does not include the nozzle (205) of the microfluidic device (200) of FIGS. 2A through 2C. The terminating chamber (304) may serve any number of purposes including acting as a micro-reaction (μ-reaction) chamber where a reaction with another fluid or a material that exists within the terminating chamber (304). In one example, the μ-reaction chamber may act as a waste fluid collector. Further, in another example, the terminating chamber (304) may serve as a drain or repository for waste fluid.

FIG. 3B is a block diagram of a microfluidic device (330) including sensors (203) to detect a high change of on/off resistance, according to an example of the principles described herein. The microfluidic device (330) of FIG. 3B includes many elements that are included within the microfluidic device (300) of FIG. 3A. The sensors (203) of FIG. 3B include sensors pairs in a first sensor pair (203-1, 203-2) and a second sensor pair (203-3, 203-4) located within the fluidic channel (202). The sensor pairs (203) may be used to detect the location of the fluid (250) within the microfluidic device (330) at a higher resolution. For example, for the first pair of sensors (203-1, 203-2), due to the distance between the first pair of sensors (203-1, 203-2), if the control logic (120) determines that the fluid (250) has passed the first impedance sensor (203-1) but not the second impedance sensor (203-2) of the first pair of sensors (203-1, 203-2), then a higher resolution as to where the fluid (250) is within the microfluidic device (330) may be determined relative to instances where the more distance exists between the sensors (203-1, 203-2) of the first pair of sensors (203-1, 203-2). The same higher resolution may be obtained at the second pair of sensors (203-3, 203-4). Thus, the pairs of sensors (203) of FIG. 3B allow the microfluidic device (330) to control the location of the fluid (250) more precisely and with a higher resolution.

Further, in the example of FIG. 3B the close proximity of the first impedance sensor (203-1) to the second impedance sensor (203-2) and the close proximity of the third impedance sensor (203-3) to the fourth impedance sensor (203-4) allows for these pairs of impedance sensors to send, via the control logic (120) a signal to a first one of the impedance sensors in the pair, and receive a resulting signal at a second one of the impedance sensors in the pair. For example, through forcing a current or voltage on the first impedance sensor (203-1), the resulting signal may be measured on the second impedance sensor (203-2) and sent to the control logic (120) from the second impedance sensor (203-2).

FIG. 3C is a block diagram of a microfluidic device (360) including sensors (230) to detect a priming flow rate, according to an example of the principles described herein. The microfluidic device (360) of FIG. 3C includes many elements that are included within the microfluidic device (300) of FIG. 3A. In some examples, it may be useful to determine how fast the fluid passively or actively travels through a passageway of the microfluidic device (360). The array of sensors (203) depicted in FIG. 3C may be located a predetermined distance between each sensor (203), and the control logic (120) may use a clock signal or other timing device to determine the flow rate of the fluid (250) through, for example, the fluidic channel (202) as well as changes in the flow rate of the fluid (250). For example, as the fluid (250) passes sensors (203-1, 203-2, 203-3, 203-4, 203-5, 203-6) in turn, the time the fluid (250) would take to do so may be used to determine the flow rate which may be measured as a volume of the fluid (250) that flows through the measured passageway of the microfluidic device (360) per unit of time and may be expressed as follows:

$$Q = \frac{dV}{dt} \qquad Eq . 1$$

where the flow rate “Q” is equivalent to the change in volume (dV) per change in time (dt).

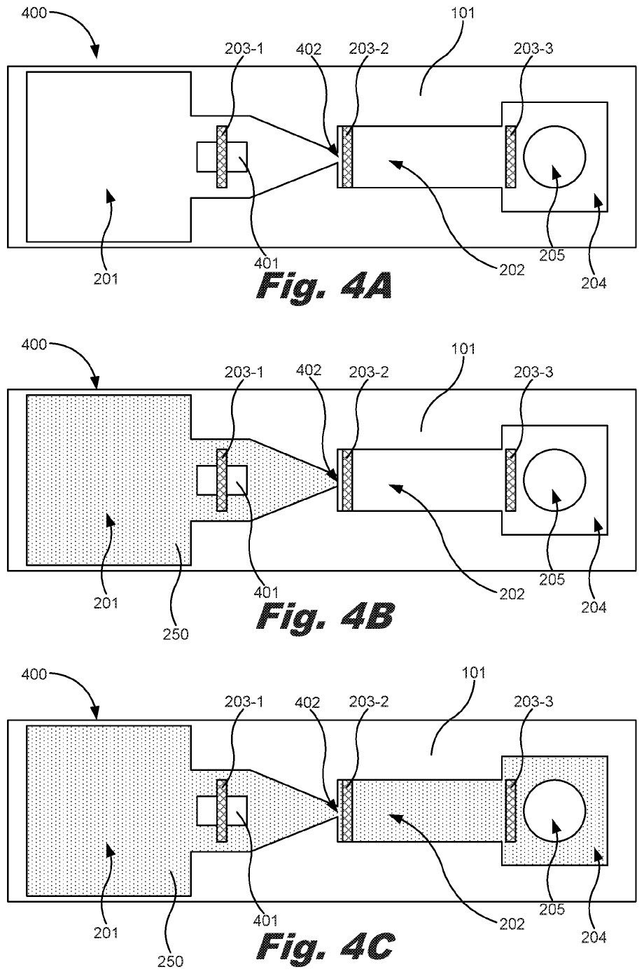

FIGS. 4A through 4C are block diagrams of a microfluidic device (400) at various levels of active priming, according to an example of the principles described herein. Active priming as defined herein utilizes the application of a pressure differential applied to the fluid to move the fluid through the passageway and prime the passageways within the microfluidic device (400). The microfluidic device (400) of FIGS. 4A through 4C includes many elements that are included within the microfluidic device (200) of FIGS. 2A through 2C and 3A through 3C. The example of FIGS. 4A through 4C include at least one pump device (401) to actively force the fluid (250) through the passageways of the microfluidic device (400) and at least one capillary break (402) to allow a discrete portion or amount of the fluid (250) to be drawn from a first passageway into another passageway.

The pump devices (401) described herein may include, for example, inertial pumps that include thermal resistive elements or piezoelectric elements, or external pump devices that are fluidically coupled to an end of the passageway to create a positive or negative pressure within the passageways to move the fluid through the passageways. The pump devices (401) may be electrically and communicatively coupled to the control logic (120) such that the control logic (120) may actuate the pump devices (401) when the fluid (250) is to be either restricted from moving within the passageways of the microfluidic device (400) or caused to move further into the passageways of the microfluidic device (400).

The capillary break (402) serves to allow a discrete portion or amount of the fluid (250) to be drawn from the reservoir (201) to the fluidic channel (202). In one example, the capillary breaks (402) described herein are formed and dimensioned to allow for at least as small as a 0.01 μL or 1 μL resolution metering of fluid (250). In other words, the fluid (250) may be drawn through the capillary break (402) at a volume of at least as small of as 0.01 μL. The capillary break (402) may be formed by decreasing the dimensions of the fluidic channel (202) to form a smaller orifice that serves to preclude movement of the fluid (250) out of the reservoir (201) and into the fluidic channel (202). An amount of the fluid (250) may be moved from the reservoir (201) and into the fluidic channel (202) through actuation of the pump device (401) that allows the fluid (250) to overcome the restraining forces provided by the capillary break (402) such that an amount of the fluid (250) enters the fluidic channel (202). In one example, the pump device (401) may be activated by the control logic (120) to meter an amount of the fluid (250) through the capillary break (402).

A first impedance sensor (203-1) may be located in the microfluidic device (400) of FIGS. 4A through 4C on or at least juxtaposition to the pump device (401) so that the control logic (120) may know when the fluid (250) has been primed within the microfluidic device (400) and reached the pump device (401) based on the impedance value detected at the first impedance sensor (203-1). In this manner, the pump device (401) may be activated when fluid (250) is present around the pump device (401).

The remainder of the impedance sensors (203-2, 203-3) of the microfluidic device (400) of FIGS. 4A through 4C function in a similar manner as described above in connection with the sensors (203-1, 203-2) described herein in connection with FIGS. 2A through 2C. Specifically, the sensors (203-2, 203-3) of the microfluidic device (400) may work singularly by providing impedance values to the control logic (120) independent of one another, and together to determine the location of the fluid (250) through their respectively detected impedance values and the differential between the two detected values.

Also, the second impedance sensor (203-2) may be used to determine when an amount of the fluid (250) has been metered from the capillary break (402). This information may be used by the control logic (120) to confirm that the pump device (401) has been activated to successfully meter the fluid (250) through the capillary break (402).

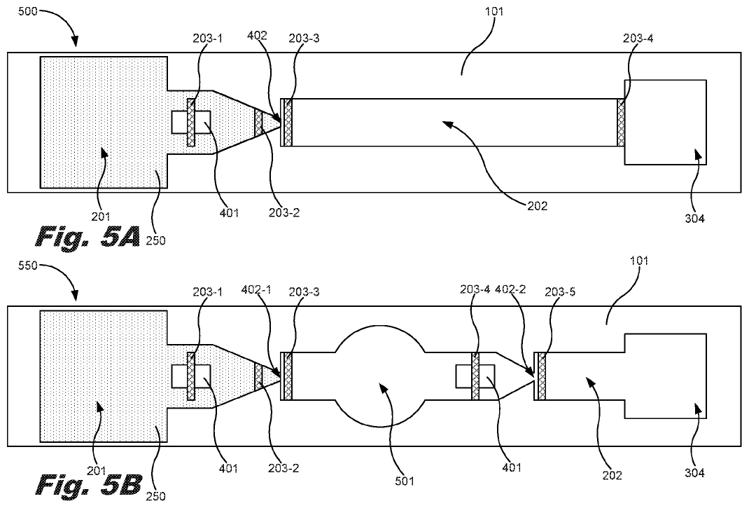

FIG. 5A is a block diagram of a microfluidic device (500) including sensors to detect single, dual, or differential active priming sense, according to an example of the principles described herein. The microfluidic device (500) of FIG. 5A may include a terminating chamber (304) as similarly described herein in connection with FIGS. 3A through 3C.

Further, the second impedance sensor (203-2) of the microfluidic device (500) may be located juxtaposition to the capillary break (402) and downstream from the pump device (401). This placement of the second impedance sensor (203-2) allows for the control logic (120) to determine if the fluid is present at the orifice of the capillary break (402) so that the pump device (401) may be activated to meter the fluid (250) into the fluidic channel (202).

Further, each of the sensors (203) in any of the examples of the microfluidic device (100, 200, 300, 330, 360, 400, 500, 550, 600, 650) described herein may work independently as individual impedance sensors (203), together as dual sensors where the detection by one impedance sensor (203) but not by another impedance sensor indicates the position of the fluid (250), and in a differential manner where the different impedances sensed by at least two separate impedance sensors (203) indicates the position of the fluid (250).

FIG. 5B is a block diagram of a microfluidic device (550) including impedance sensors (203) to detect a boundary of fluid within a passageway of the microfluidic device (550), according to an example of the principles described herein. The microfluidic device (550) includes many elements that are included within the microfluidic device (500) of FIG. 5A. The microfluidic device (550) of FIG. 5B may further include a micro-reaction (μ-reaction) chamber (501) in which the fluid (250) is allowed to react. In one example, the μ-reaction chamber (501) may be a passageway within the microfluidic device (550) where the fluid (250) is allowed to remain for a period of time. In another example, the μ-reaction chamber (501) may be a passageway within the microfluidic device (550) where the fluid (250) is reacted with another fluid or a chemical already present within the μ-reaction chamber (501) may react with the fluid (250). Still further, the μ-reaction chamber (501) may be a passageway within the microfluidic device (550) where the fluid (250) is subjected to a physical change such as changing the fluid's (250) temperature through the use of a cooling or heating device, mixing of the fluid (250), exposure of the fluid (250) an electromagnetic field, other physical processes or combinations thereof. Even still further, the μ-reaction chamber (501) may be a passageway within the microfluidic device (550) where the fluid (250) is subjected to a combination of the above-described chemical and physical processes. In this manner, the μ-reaction chamber (501) causes the microfluidic device (550) to serve as a lab-on-chip device where the fluid (250) is subjected to chemical and physical processes, and characteristics of the processed fluid (250) may be measured.

The microfluidic device (550) of FIG. 5B also includes a second pump device (401-2) and a second capillary break (402-2) in addition to a first pump device (401-1) and a first capillary break (402-1). The second capillary break (402-2) may serve to assist in retaining the fluid (250) within the μ-reaction chamber (501) so that the fluid (250) may undergo the chemical and physical processes described herein without draining to the terminating chamber (304). Further, the second pump device (401-2) may be activated by the control logic (120) to force a processed fluid (250) from the μ-reaction chamber (501) and into the fluidic channel (202) and terminating chamber (304) after, for example, the characteristics of the processed fluid (250) are measured.

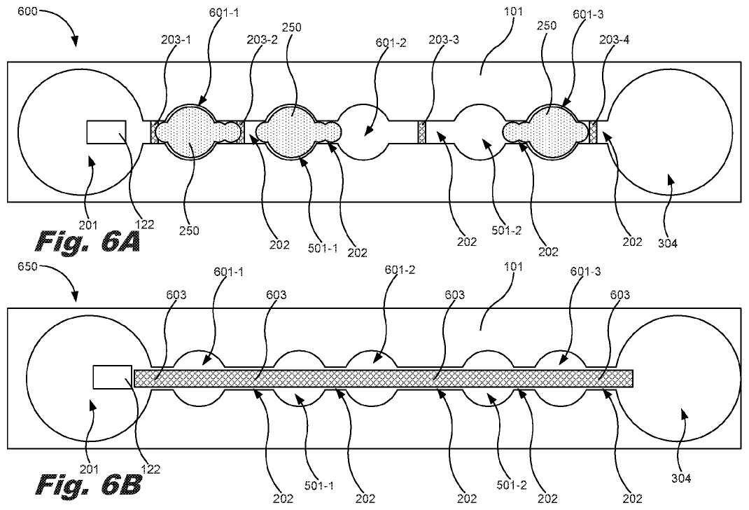

FIG. 6A is a block diagram of a microfluidic device (600), according to an example of the principles described herein. The microfluidic device (600) includes many elements that are included within the microfluidic devices (100, 200, 300, 330, 360, 400, 500, 550) described herein. Further, the microfluidic device (600) of FIG. 6A may include at least one staging chamber (601-1, 601-2, 601-3, collectively referred to herein as 601). The staging chambers (601) may be defined in the die (101) before a respective μ-reaction chamber (501-1, 501-2, collectively referred to herein as 501) and may be separated from its respective μ-reaction chamber (501-1, 501-2) by a fluidic channel (202).

The fluidic channels (202) that separate the staging chambers (601) and the μ-reaction chambers (501) may each include an impedance sensor (203-2, 203-3) that is used to determine if a volume of the fluid (250) has reached the location of the respective impedance sensor (203-2, 203-3). This is helpful in providing feedback from the impedance sensor (203-2, 203-3) regarding the position of the fluid (150) to the control logic (120), and allowing the control logic (120) to activate a pump device (401) or an external pump device based on the feedback. Specifically, the pump device (401) or external pump device may be activated by the control logic (120) to create a pressure differential in order to retain the fluid (250) at a specific point within the microfluidic device (600).

Specifically, the pump device (401) or external pump device may be activated by the control logic (120) to create a pressure differential in order to move the fluid (250) within the passageways of the microfluidic device (600). In the examples described herein, the fluid (250) may be retained at a specific point within a staging chamber (601), upstream from the associated impedance sensor (203), and not flowing into the μ-reaction chamber (501). For example, a pump device (401) or external pump device may be included in the microfluidic device (600) and may be activated to retain the fluid (250) within the first staging chamber (601-1) without passing the second impedance sensor (203-2) and into the first μ-reaction chamber (501-1). This may be achieved by the control logic (120) causing the second impedance sensor (203-2) to sense the impedance at the second impedance sensor (203-2) to determine if the fluid has flowed past the second impedance sensor (203-2). If the fluid has touched or passed the second impedance sensor (203-2), then the control logic (120) may activate the pump device (401) or external pump device to cause the fluid to move back upstream into the first staging chamber (601-1) and upstream from the second impedance sensor (203-2). If it is desired that the fluid should be moved into the first μ-reaction chamber (501-1) from the first staging chamber (601-1), then the control logic (120) may either cause the pump device (401) or external pump device to remove the differential pressure to allow the fluid (250) to flow into the first μ-reaction chamber (501-1) through capillary forces, or may cause the pump device (401) or external pump device to provide a downstream differential pressure to force the fluid (250) to flow into the first μ-reaction chamber (501-1). The fourth impedance sensor (203-4) may be used in a manner similar to the uses described herein for the second impedance sensor (203-2) and the third impedance sensor (203-3) when it is desired that the fluid (250) is to either be kept form entering or should be allowed to enter the terminating chamber (304).

FIG. 6B is a block diagram of a microfluidic device (650) including an extended sensor (603), according to an example of the principles described herein. The microfluidic device (650) includes many elements that are included within the microfluidic devices (100, 200, 300, 330, 360, 400, 500, 550, 600) described herein. Further, the microfluidic device (650) includes the extended sensor (603). In the example of FIG. 6B, instead of multiple “point” impedance sensors (203) along the length of the microfluidic device (650) and within the various passageways between the reservoir (201) and nozzle chamber (204) or terminating chamber (304), the impedance sensor (603) may be a longer sensor that extends, for example, through a plurality of passageways of the microfluidic device (650) such as a staging chamber (601), a μ-reaction chamber (501), a fluidic channel (202), a reservoir (201), a nozzle chamber (204) or terminating chamber (304), and combinations thereof.

In this example, the impedance sensor (603) may include a conductive plate of an aspect ratio similar to the passageways such as the fluidic channel (202). The conductive plate forms a capacitor that produces an analog measurement proportional to how much fluid is in contact with the conductive plate. In this example, the fluid may form an at least partially conductive path to or from the plate, and an analog signal proportional to the amount of fluid in contact with the conductive plate may be observed. The control logic (120) may activate the impedance sensor (603) to receive a single measurement. This single measurement may be an analog signal that correlates to how much of the passageways are filled with the fluid (250). The extended impedance sensor (603) may provide for a simpler microfluidic device (650) that is easier to manufacture due to the single circuit created between the control logic (120) and the impedance sensor (603). Further, in some instances mismatches and electrical parasitics that may exist between a plurality of impedance sensors (203) as depicted in other examples herein is eliminated. In an example, instead of a single, extended sensor, the impedance sensor (603) may include an array of a plurality of electrodes that are aligned with respect to one another. In this example, a pseudo-analog signal may be detected by the array.

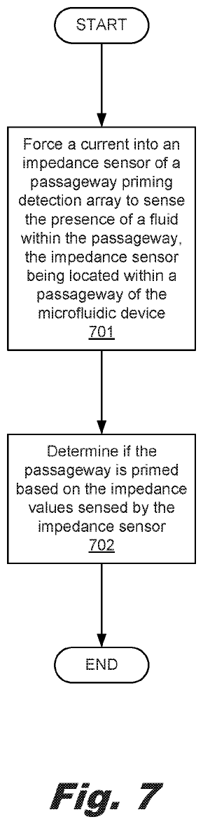

FIG. 7 is a flowchart showing a method (700) of detecting priming of a microfluidic device (100, 200, 300, 330, 360, 400, 500, 550, 600, 650, collectively referred to herein as 100), according to an example of the principles described herein. The method (700) may include forcing (block 701) a current into an impedance sensor (103, 203, 603, collectively referred to herein as 103) of a passageway priming detection array to sense the presence of a fluid (250) within the passageway. The passageway priming detection array may include any number of impedance sensors (103). The passageway may include any architecture defined within the die (101) of the microfluidic device (100) described herein including the fluidic channels (102, 202, collectively referred to herein as 102), the reservoir (201), the nozzle chamber (204), the nozzle (205), the terminating chamber (304), the capillary break (402), the μ-reaction chamber (501), staging chamber (601) other architecture elements, and combinations thereof. At least one impedance sensor (103) is located within at least one passageway of the microfluidic device (100).

The method (700) may also include determining (block 702) if the passageway is primed with the fluid (250) based on the impedance values sensed by at least one impedance sensor (103) within the microfluidic device (100). The control logic (120) activates at least one impedance sensor (103) to detect the impedance of the fluid (i.e., air or non-air fluid) at the location of the impedance sensor (103), and receives from the impedance sensor (103) an impedance value that defines whether the fluid (250) exists at that impedance sensor (103).

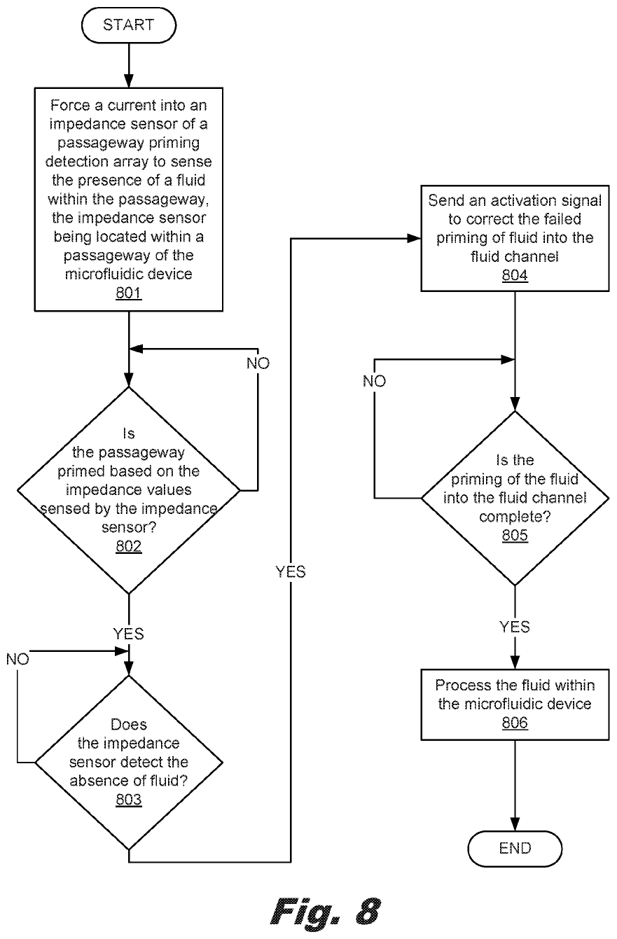

FIG. 8 is a flowchart showing a method (800) of detecting priming of a microfluidic device (100), according to an example of the principles described herein. The method (800) may include forcing (block 801) a current into an impedance sensor (103) of a passageway priming detection array to sense the presence of the fluid (250) within the passageway, and determining (block 802) if the passageway is primed with the fluid (250) based on the impedance values sensed by at least one impedance sensor (103) within the microfluidic device (100). In response to a determination that the passageway is not primed (block 802, determination NO), the method (800) may loop back to the outset of block 802 so that the impedance is continuously monitored and sensed to determine when the priming has competed.

In response to a determination that the passageway is primed (block 802, determination YES), then the impedance may be continuously monitored and sensed at the at least one impedance sensor (100), and it may be determined (block 803) whether the impedance sensor (103) detects the absence of fluid (250) in the passageway once the fluid (250) has been detected as primed into the passageway. The absence of the fluid (250) in this situation indicates a failed priming of fluid (250) into the passageway. In response to a determination that the impedance sensor (103) does not detect the absence of fluid (250) in the passageway (block 803, determination NO), then the method may loop back to the outset of block 803 so that the impedance is continuously monitored and sensed in order to determine the point at which the passageway is primed. In response to a determination that the impedance sensor (103) does detect the absence of fluid (250) in the passageway (block 803, determination YES), then the method may proceed wo block 804 where an activation signal is sent (block 804) form the control logic (120) to, for example, a pump device (401) or an external pump device, to correct the failed priming of the fluid into the passageway. The activation signal from the control logic (120) includes a signal to cause the pump device (401) or an external pump device to move fluid (250) into the passageway via an orifice such as the reservoir (201).

The method may also include determining (block 805) when the priming of fluid into the fluidic channel is complete. In response to a determination that the priming of fluid (250) into the passageway is not complete (block 805, determination NO), the method may loop back to the outset of block 805 so that the impedance is continuously monitored and sensed to determine when the priming has competed. In response to a determination that the priming of fluid (250) into the passageway is complete (block 805, determination YES), then the control logic may send instructions to a number of measuring and processing devices that may be included within the microfluidic device (100) to allow the microfluidic device (100) to process (block 806) the fluid within the microfluidic device (100). Processing the fluid within the microfluidic device (100) may include detecting any chemical or physical characteristic of the fluid (250), subjecting the fluid (250) to a chemical or physical process as described herein, or combinations thereof.

Aspects of the present system and method are described herein with reference to flowchart illustrations and/or block diagrams of methods, apparatus (systems) and computer program products according to examples of the principles described herein. Each block of the flowchart illustrations and block diagrams, and combinations of blocks in the flowchart illustrations and block diagrams, may be implemented by computer usable program code. The computer usable program code may be provided to a processor of a general-purpose computer, special purpose computer, or other programmable data processing apparatus to produce a machine, such that the computer usable program code, when executed via, for example, the control logic (120) of the microfluidic device (100) or other programmable data processing apparatus, implement the functions or acts specified in the flowchart and/or block diagram block or blocks. In one example, the computer usable program code may be embodied within a computer readable storage medium; the computer readable storage medium being part of the computer program product. In one example, the computer readable storage medium is a non-transitory computer readable medium.

The specification and figures describe a microfluidic device. The microfluidic device may include an impedance sensor located within a fluidic priming orifice within a fluidic channel of the microfluidic device, and control logic. The control logic forces a current into the impedance sensor to sense the presence of a fluid within the fluidic channel at the locations of the impedance sensor, and determines if the fluid is primed into the fluidic channel based on the impedance values sensed by the impedance sensor.

The microfluidic devices and methods described herein provide in-situ confirmation of fluidic priming for lab-on-chip microfluidic applications. Further, the microfluidic devices and methods described herein provide feedback to the control logic of the microfluidic devices to resolve any priming failures or malfunctions. The devices, systems, and methods described herein also reduce fluid test time by replacing passive priming wait-times with real-time priming feedback, and detect the presence of air in microfluidic systems that may decrease measurement accuracy. Still further, the devices, systems, and methods described herein detect an end of fluid supply (i.e., empty reservoir) condition for micro-titration and other fluid processing applications.

The preceding description has been presented to illustrate and describe examples of the principles described. This description is not intended to be exhaustive or to limit these principles to any precise form disclosed. Many modifications and variations are possible in light of the above teaching.

What is claimed is:

1. A microfluidic device, comprising:
an impedance sensor located within a fluidic priming orifice within a fluidic channel of the microfluidic device; and
control logic to:
force a current into the impedance sensor to sense the presence of a fluid within the fluidic channel at the locations of the impedance sensor; and
determine if the fluid is primed into the fluidic channel based on the impedance values sensed by the impedance sensor.

2. The microfluidic device of claim 1, wherein the control logic monitors for at least a second time, the impedance sensed at the impedance sensor.

3. The microfluidic device of claim 2, wherein the control logic is to:
determine if the impedance sensor detects the absence of fluid in the fluidic channel once the fluid has been detected as primed into the fluidic channel, the absence of the fluid indicating a failed priming of fluid into the fluidic channel; and
send an activation signal to correct the failed priming of fluid into the fluidic channel.

4. The microfluidic device of claim 3, wherein the activation signal to correct the failed priming of fluid into the fluidic channel comprises a signal to cause a pumping device to move fluid into the fluidic channel.

5. The microfluidic device of claim 1, wherein the impedance sensor comprises a single impedance sensor comprising a conductive plate with a length of at least a portion of the fluidic channel, and wherein the single impedance sensor, when actuated, provides an analog signal that correlates with an amount of fluid within the fluidic channel.

6. The microfluidic device of claim 1, wherein the control logic is to:
determine when the priming of fluid into the fluidic channel is complete; and
in response to a determination that the priming of fluid into the fluidic channel is complete, instruct the processing of the fluid within the microfluidic device.

7. A system for detecting priming of a microfluidic device, comprising:
a fluidic channel priming detection array comprising at least one impedance sensor located within a fluidic channel of the microfluidic device; and
control logic to:
force a current into the impedance sensor to sense the presence of a fluid within the fluidic channel;
determine if the fluidic channel is primed based on the impedance values sensed by the impedance sensor; and
send an activation signal to correct a failed priming of fluid into the fluidic channel in response to a determination that the fluidic channel is not primed.

8. The system of claim 7, wherein the control logic is to:
monitor the impedance sensed about the impedance sensor at least a second time; and
determine if the impedance sensor detects the absence of fluid in the fluidic channel once the fluid has been detected as primed into the fluidic channel, the absence of the fluid indicating a failed priming of fluid into the fluidic channel.

9. The system of claim 8, wherein the activation signal to correct the failed priming of fluid into the fluidic channel comprises a signal to cause a pumping device to move fluid into the fluidic channel via the orifice.

10. The system of claim 7, wherein the at least one impedance sensor of the fluidic channel priming detection array comprises at least:
a first impedance sensor located at a first position at a fluidic priming orifice within a fluidic channel of the microfluidic device; and
a second impedance sensor located at a second position within the fluidic priming orifice within the fluidic channel downstream relative to the first impedance sensor,
wherein the first impedance sensor and the second impedance sensor detect the presence of the fluid at relative locations of the first impedance sensor and the second impedance sensor.

11. The system of claim 7, wherein the at least one impedance sensor of the fluidic channel priming detection array comprises a conductive plate with a length of at least a portion of the fluidic channel, and wherein the single impedance sensor, when actuated, provides an analog signal that correlates with an amount of fluid within the fluidic channel.

12. The system of claim 7, wherein the control logic is to:
determine when the priming of fluid into the fluidic channel is complete; and
in response to a determination that the priming of fluid into the fluidic channel is complete, instruct the processing of the fluid within the microfluidic device.

13. A method of detecting priming of a microfluidic device, comprising:
forcing a current into an impedance sensor of a fluidic channel priming detection array to sense the presence of a fluid within the fluidic channel, the impedance sensor being located within a fluidic channel of the microfluidic device; and
determining if the fluidic channel is primed based on the impedance values sensed by the impedance sensor.

14. The method of claim 13, further comprising:
continuously monitoring the impedance sensed at the impedance sensor;
determining if the impedance sensor detects the absence of fluid in the fluidic channel once the fluid has been detected as primed into the fluidic channel, the absence of the fluid indicating a failed priming of fluid into the fluidic channel; and
sending an activation signal to correct the failed priming of fluid into the fluidic channel,
wherein the activation signal to correct the failed priming of fluid into the fluidic channel comprises a signal to cause a pumping device to move fluid into the fluidic channel via the orifice.

15. The method of claim 13, further comprising:
determining when the priming of fluid into the fluidic channel is complete; and
in response to a determination that the priming of fluid into the fluidic channel is complete, instructing the processing of the fluid within the microfluidic device.
